(19) United States
(12) Patent Application Publication
(10) Pub. No.: US 2007/0225932 A1
(43) Pub. Date: Sep. 27, 2007

(54) Methods, systems and computer program products for extracting paroxysmal events from signal data using multitaper blind signal source separation analysis

(76) Inventor: Jonathan Halford, Mount Pleasant, SC (US)

Correspondence Address: JENKINS, WILSON, TAYLOR & HUNT, P. A., SUITE 1200, UNIVERSITY TOWER, 3100 TOWER BOULEVARD, DURHAM, NC, 27707, US

(21) Appl. No.: 11/701,770

(22) Filed: Feb. 2, 2007

Related U.S. Application Data
(60) Provisional application No. 60/764,624, filed on Feb. 2, 2006.

(51) Int. Cl. G01D 1/00; A61B 5/04

(52) U.S. Cl. 702/127; 600/544

(57) ABSTRACT

Methods, systems and computer program products for extracting paroxysmal events from signal data using multitaper blind signal source separation analysis are disclosed. According to one method, a signal data set including signal data from a plurality of channels is received. Blind signal source separation analysis is repeatedly performed on different time limited segments across the signal data in a multitaper method to extract a plurality of components indicative of paroxysmal events from the signal data. The components indicative of paroxysmal events are presented to a user.

### RELATED APPLICATIONS

This application claims the benefit of U.S. Provisional Patent Application Ser. No. 60/764,624, filed Feb. 2, 2006; the disclosure of which is incorporated herein by reference in its entirety.

### TECHNICAL FIELD

The subject matter described herein relates to analyzing signal data from multiple sources. More particularly, the subject matter described herein relates to methods, systems, and computer program products for extracting paroxysmal events from signal data using multitaper blind signal source separation analysis.

### BACKGROUND

Introduction

In many fields, it is desirable to identify or extract discreet events from signal data collected from multiple sources. For example, in the field of electroencephalography, multiple sensors are placed on a patient's scalp and record electrical signals generated by the patient's brain. It is desirable to parse these signals to identify portions of the signals indicative of events, such as eye blink events. Other fields in which it may be desirable to identify discreet events from signal data include seismography, where it is desirable to identify seismic event signatures from signal data collected at different locations and sonography, where it is desirable to identify a signature sound from signals collected by different sensors.

In the field of electroencephalography, advancement in digital electroencephalography provides new opportunities but also new challenges. The dream of many clinical neurophysiologists is to be able to visualize and analyze many of the important electrical signals occurring in the human brain. Unfortunately, there is no safe system which will allow a clinical neurophysiologist to place as many electrodes as he or she would like anywhere in the brain. The human brain makes measurement of its electrical potentials difficult due to its compact size and elaborate protection by bone and other tissues. Due to this limitation, most of the electrical signals recorded from the brain, particularly when recorded from the scalp, provide limited information due to several technical limitations. First, these signals (particularly those recorded from the scalp) are confounded by various noncerebral artifacts. Secondly, signals from many different brain sources are mixed together and localization of their origin is difficult. Third, many electrocortical signals which may be of clinical relevance are of low amplitude and therefore difficult to discern from the intermixed noise and other higher-amplitude brain potentials. But despite these limitations, the recording of scalp EEG is clinically useful. Could EEG data be made more useful if some of these limitations could be minimized?

One new challenge is the vast quantities of EEG which can now be recorded digitally while a subject is in the hospital or during ambulatory monitoring. These large EEG datasets probably contain a great deal of untapped clinically relevant information. But, due to the time it takes an electroencephalographer to review a prolonged EEG monitoring dataset, much of this data is not being visualized by electroencephalographer, much less used to make clinical decisions. Thoroughly reviewing 24 hours of EEG data, using conventional techniques (viewing 10 seconds of EEG data at a time), takes most electroencephalographers at least 20 minutes. Some patients have inpatient monitoring for many days or even weeks. Ambulatory monitoring currently is done for 1-2 days, but this may be extended as memory storage chips, battery technologies, and scalp electrode application techniques improve. More advanced electromagnetic signal acquisition techniques, such as intracranial EEG monitoring, high-density scalp EEG monitoring, and magnetoencephalography produce even more signals that need to be reviewed. New techniques are needed to help electroencephalographers sift rapidly through these large volumes of clinical signal data.

The Event Extraction and Presentation Paradigm

Many of the most interesting and clinically relevant EEG patterns are paroxysmal events. Clinically relevant paroxysmal events include epileptiform spikes and sharp waves, bursts of slowing, sleep morphology (such as vertex waves and sleep spindles), and seizures. For purposes of signal analysis, a paroxysmal event may be defined as a pattern consisting of increased electrical activity of a particular shape with a beginning and an end which originates from a specific spatial distribution. In EEG applications, many paroxysmal events have particular characteristics which are unique, including average length, shape, peak spectral frequency, and scalp distribution. If a computer could be instructed to capture all paroxysmal events within an EEG record, these events could potentially be cataloged and categorized automatically and then presented to the electroencephalographer in an organized scheme. This could enable an electroencephalographer to come to a good understanding of an important aspect of the EEG record without having to review all the raw EEG data.

Much of advanced signal analysis research has focused on techniques which are designed to replace the clinical neurophysiologist—such as automated interpretation. The idea is to either (1) catalogue all the characteristics the a neurophysiologist uses to judge abnormality and apply those to a dataset yielding a clinical judgment or (2) use a neural network approach in which there is a training signal provided by a seasoned clinician to a neural network which trains it to yield a clinical judgment. This effort has produced mixed results and overall no system has been produced which a significant number clinical neurophysiologist would rely on to make clinical decisions. But an important question is why is it desirable to remove the clinical neurophysiologist from the clinical decision making progress? The human mind possesses much more computing power than even the largest computer cluster in existence now. If the goal is to make clinical epilepsy care cheaper, then removing the computer might be an option at some point. But if the goal is to improve interpretation of electroencephalographic data, the best combination would be for a computing system and a human expert to work in tandem. This process is depicted (in very general form) in FIG. 1.

In this paradigm, the computer would not be required to render clinical judgment, only to aid the neurophysiologist. The central question in this paradigm is how can a computer best aid a neurophysiologist? One way for a computer to help a neurophysiologist understand a very large dataset of raw neurophysiology signals is for the computer to be able to detect, isolate, sort and represent aspects to a neurophysiology dataset which a human neurophysiologist would find interesting. What humans find interesting are events. Despite the fact that human lives unfold to them continuously, when humans converse, they more often than not discuss events. For the purposes of explaining the examples herein, an event is defined as a subset of the signal-versus-time dataset which:
(1) begins and ends at certain time points
(2) occurs in a particular subset of the data channels (has a particular spatial distribution)
(3) has a higher average amplitude then the other signal activities within its subset of data channels

To clarify this definition of the word event, it is also added that:
(1) An event can be of any length. (A specific length would be assigned to a detected event, but events could be of any length that the computer system was designed to detect)
(2) The precise signal morphology is not rigorously specified except that it has a higher signal amplitude than the signal data before and after it within its spatial distribution.

This is consistent with the way humans experience events in our lives. Humans have sensory signals coming to them at all of the time through their various senses in a continuous fashion. Yet humans perceive distinct noteworthy events within those continuous signals coming from their senses. Events are distinguished as noteworthy because they stand out in some way from the background signals.

It is believed to be likely that clinical neurophysiologists and other scientists working in other fields would enjoy using a computer system that scoured an electrocorticography dataset or other signal-versus-time dataset for events, collected these events, categorized these events, and presented these events to the viewer in an visually pleasing, accurate, and intuitive scheme. Then the clinician or scientist would not have to look through hours of neurophysiologic data, but would look through an organized and sorted list of events.

The goal would be to create a process similar to routine clinical magnetic resonance imaging (MRI) data analysis and presentation. A radiologist does not review the copious amounts of raw signal data which are produced by a MRI machine during image acquisition. The MRI computers automatically process this signal data and present the clinically relevant information in an organized and intuitive scheme to the radiologist. The difference would be that this scheme, as applied to electroencephalographic or magnetoencephalographic data, would be a method for studying spontaneous neurophysiological data in this way. This is an important distinction, since studying spontaneous neurophysiological data is in many ways much more difficult than studying evoked potentials. A MRI scan, in its most basic description, is a type of neurophysiologic evoked potential. The MRI machine creates an appropriate environment for its type of signal acquisition by the use of a high field-strength fixed magnet. Next, radio frequency signals are transmitted into the human body. Finally, the human body produces signals in response and through signal averaging techniques (using gradient-type magnetic fields), isolation of signals of interest from all of the radio frequency signals provoked from the human body is achieved. In evoked potential analysis, the computer does not have to pay close attention to all of the incoming signal data in its analysis since (1) it is instructed on when the relevant data is going to arrive, (2) it is instructed on where it is going to arrive from (approximately), and (3) it is allowed to average the signal data in its analysis. In analyzing spontaneous signals, (1) the interpreting computer or neurophysiologist does not know when the most important or relevant signals are going to arrive, (2) the interpreting computer or scientist does not know where within the recording field these signals are going to come from, and (3) data analysis preferably does not involve signal averaging since signal averaging destroys potentially irretrievable information. Therefore, if computer software is to be created that might be able to best analyze spontaneous neurophysiological or other signal data, it should be assumed that this computing system will be very computationally intensive. In fact, any computational technique which claims to be able to do this and is not very computationally intensive should be viewed with skepticism.

### SUMMARY

Methods, systems and computer program products for extracting paroxysmal events from signal data using multitaper blind signal source separation analysis are disclosed. According to one method, a signal data set including signal data from a plurality of channels is received. Blind signal source separation analysis is repeatedly performed on different time limited segments across the signal data in a multitaper method to extract a plurality of components indicative of paroxysmal events from the signal data. The components indicative of paroxysmal events are presented to a user.

The subject matter described herein includes a technique for the extraction of events from signal data using blind signal source separation analysis. Using one exemplary technique, events may be extracted from EEG data using a type of blind signal source separation analysis, referred to as independent component analysis (ICA). The purpose of this technique is to attempt to design a computer method for detecting, characterizing, sorting, and presenting neurophysiological events. This is a method which is predicated on the availability of enormous computing power. It is assumed that, due to the rapid expansion of available computing power in the ongoing computer revolution, limitations in computing power which are present today probably will not be limitations in the future. The preparation of computer software to use this future computing power to better understand the brain's electrocortical signals must begin now. Software development takes time. If development is delayed until the computing power is readily available, this will delay the ability to use this computing power effectively for research once it arrives. This technique for using blind signal source separation to understand signal events is designed to overcome the limitations of typical electrocortical signal analysis techniques that are listed above. But, to reiterate, these limitations are:
(1) Most current techniques use some sort of signal averaging, which limits information available for analysis of spontaneous neurophysiological signal events.
(2) Most current techniques do not allow for the detection of single spontaneous low-amplitude neurophysiological event signals.
(3) Most current techniques do not allow the analysis of large quantities of EEG data and the presentation to neurophysiologists except through simple techniques such as frequency power analysis or amplitude integration.
(4) Most current techniques do not separate out artifactual signals from electrocortical signals well.
   Independent Component Analysis (ICA) and its Limitations

ICA is one method for performing blind signal source separation and extracting individual source signals from a mixture of signals [Bell, 1995#59]. ICA theory has received attention from several research communities including machine learning, neural networks, statistical signal processing and Bayesian modeling. ICA and other types of blind signal source separation have application at the intersection of many science and engineering disciplines concerned with understanding and extracting useful information from data as diverse as neuronal activity and brain images, bioinformatics, communications, the world wide web, audio, video, sensor signals, or time series. ICA is being applied to the analysis of EEG data in several ways. ICA has been used to study the source signals underlying auditory evoked potentials [Makeig, 1997 #54] and visual evoked potentials [Makeig, 2002#55]. ICA has been used to remove artifacts from EEG recordings [Jung, 2000#56] to analyze epileptiform spikes [Kobayashi, 2001#57] and to analyze absence seizures [McKeown, 1999#60].

Blind signal source separation (BSSS) methods such has ICA are an ideal method for achieving EEG signal preprocessing through deconstruction into events for these reasons:

(1) ICA deconstructs signal data into lists of signal patterns. Each of these signal patterns, called independent components in ICA terminology, contains events which occur in only one particular number of signal channels detected at a particular area of the scalp (2) ICA does not involve signal averaging and therefore minimizes, in its very nature, the amount of neurophysiologic signal information that is lost in the data analysis process. (3) ICA is very computationally intensive and therefore is theoretically promising as a method for attempting to do the complex tasks which a clinical neurophysiologist's mind does.

The limitations of conventional ICA (and other current algorithms for performing BSSS) for isolating events within neurophysiologic datasets are twofold. First, conventional ICA does not provide a measure of the length of events that are isolated. An EEG dataset is decomposed into a number of independent components equal to the number of recording scalp electrodes. All of these independent components have the length of the original dataset, even if the events that they contain are of various smaller lengths. Essentially, the length of the independent components produced is decided before the computer analysis a priori by the analyzer despite the individual lengths of the neurophysiological events that the analyzed EEG dataset may contain. Secondly, conventional ICA is limited to isolating a number of event types equal to the number of scalp electrodes. If more event types than scalp electrodes are present within a dataset, some events may not be isolated. This is called the “over-completeness problem” of ICA. For example, in a long EEG recording, if there are over 25 different types of paroxysmal events (each with a different source location on the scalp) present in the data but only 20 scalp electrodes, some types of events will not appear in the ICA analysis (except as noise). This is particularly problematic when analyzing long neurophysiologic datasets acquired with the standard clinical electrode placement, providing only approximately 20 channels. In long segments of EEG data, it is likely that there are more than 20 different types of paroxysmal events present (especially since many types signal events will be artifacts of noncerebral origin which abound within neurophysiologic datasets).

This problem can be illustrated with an analogy. Signal source separation analysis techniques such as ICA attempt to solve what is called the “cocktail party problem”. This problem is briefly stated below:
Suppose there is a cocktail party which contains N number of people in it which are all conversing at once. On the ceiling there are N number of microphones. Each microphone is in a different location. Each signal from each microphone contains a mixture of the signals produced by N number of people talking in the cocktail party. Given that only the signal from each microphone and each microphone's location within the room is known, can those signals be used to calculate (1) the signal produced by each person's voice and (2) each person's approximate location in the room?
   Due to the brilliant work of mathematicians and engineers over the last decade, this problem is being solved. Given the signals from the microphones and the locations of the microphones in the room are known, the original voices and their approximate locations can now be calculated. But, unfortunately, the cocktail party problem confronting neurophysiologists and other scientists is much more complicated than the problem above. The brain and other signal sources in the natural world are, obviously, very complex and there are usually more people (source signals) in the cocktail party than there are microphones. (signal detectors), leading to a big “over-completeness” problem. Also, these persons in the cocktail party do not necessarily stand still—sometimes they walk around the room. To make matters worse, people come and go at the party, so the number of sources may change. All of this complicates the problem tremendously and requires that signal source separation, as computationally intensive as it is, be made more complex and more computationally intensive if it is to attempt to solve the problem and provide a description and location of source signals.

The subject matter described herein for extracting paroxysmal events from signal data using blind signal source separation analysis can be implemented using a computer program product comprising computer-executable instructions embodied in a computer-readable medium. Exemplary computer readable media suitable for implementing the subject matter described herein include disk memory devices, chip memory devices, programmable logic devices, application specific integrated circuits, and downloadable electrical signals. In addition, a computer program product that implements the subject matter described herein may be located on a single device or computing platform or may be distributed across multiple devices or computing platforms. Due to the computational requirements of repetitive blind signal source separation analysis described herein, the software that performs this analysis may be distributed across multiple computing platforms to reduce computation time.

### BRIEF DESCRIPTION OF THE DRAWINGS

Preferred embodiments of the subject matter described herein will now be explained with reference to the accompanying drawings, of which:

FIG. 1 is a flow diagram illustrating a goal in processing EEG data to facilitate interpretation by a clinician;

FIG. 2 is a diagram of a signal representing a paroxysmal event of interest and overlapping time windows or segments used to extract the event from signal data using multitaper blind signal source separation analysis according to an embodiment of the subject matter described herein;

FIG. 3 is a diagram of a signal representing a paroxysmal event of interest recorded on a different channel than the signal of FIG. 2 and overlapping time windows or segments are used to extract the event from signal data using multitaper blind signal source separation analysis according to an embodiment of the subject matter described herein;

FIG. 4 is a diagram of a signal representing a paroxysmal event of interest and plural overlapping time windows or segments are used to extract the event from signal data using multitaper blind signal source separation analysis according to an embodiment of the subject matter described herein;

FIG. 5 is a graph of paroxysmal event indices (PEls) computed for different time segment or window lengths with different zone lengths using multitaper blind signal source separation analysis according to an embodiment of the subject matter described herein;

FIG. 6 is a graph of EEG data displayed in AP bipolar montage before applying multitaper blind signal source separation analysis to the EEG data;

FIG. 7 is the same graph of PEls illustrated in FIG. 5 with paroxysmal events detected using multitaper blind signal source separation analysis according to an embodiment of the subject matter described herein identified by letters A-G;

FIG. 8 includes graphs of independent components and corresponding scalp distributions detected using multitaper blind signal source separation analysis according to an embodiment of the subject matter described herein;

FIG. 9 is a graph of the same EEG data illustrated in FIG. 6 with events identified using multitaper blind signal source separation analysis according to an embodiment of the subject matter described herein highlighted with shading and labeled with letters A-G;

FIG. 10 is a graph of EEG data displayed in transverse bipolar montage with events identified using multitaper blind signal source separation analysis according to an embodiment of the subject matter described herein highlighted by shading and letters A-G;

FIG. 11 includes graphs of different zones of independent components detected using multitaper blind signal source separation analysis and a cross-multiplied version of the signals according to an embodiment of the subject matter described herein;

FIG. 12 is a graph of a Gaussian-derived function used to minimize the number of independent components generated for the same event according to an embodiment of the subject matter described herein;

FIGS. 13 and 14 are diagrams of a component technique for performing independent component analysis according to an embodiment of the subject matter described herein;

FIG. 15 is a graph of graph of a sign change factor (SCF) used to decrease the effect of frequency-based bias of independent components on PEI values according to an embodiment of the subject matter described herein;

FIG. 16 is a graph of an independent component and a corresponding scalp distribution used to illustrate a redundancy reduction method according to an embodiment of the subject matter described herein;

FIG. 17 is a graph of an independent component and a corresponding scalp distribution with an IDF value greater than one and that therefore does not require inversion according to an embodiment of the subject matter described herein;

FIG. 18 is a graph of an independent component that has been inverted and a corresponding scalp distribution according to an embodiment of the subject matter described herein;

FIG. 19 is a flow diagram illustrating an exemplary application of multitaper blind source signal selection according to an embodiment of the subject matter described herein;

FIG. 20 is a block diagram illustrating an exemplary system of extracting paroxysmal events from signal data using multitaper blind signal source separation analysis according to an embodiment of the subject matter described herein;

FIG. 21 is a flow chart illustrating an exemplary process for extracting paroxysmal events from signal data using multitaper blind signal source separation analysis according to an embodiment of the subject matter described herein; and

FIG. 22 is a graphical illustration of multitaper blind signal source separation analysis according to an embodiment of the subject matter described herein.

### DETAILED DESCRIPTION

The subject matter described herein includes methods, systems, and computer program products for identifying paroxysmal events in signal data using multitaper blind signal source separation analysis. In one implementation, the type of blind signal source separation analysis that is used is independent component analysis. In this technique, independent component analysis is applied repetitively with varying window sizes in a multitaper method to signals in a signal dataset to produce independent components. Independent components that possess a paroxysmal appearance are selected and stored. Redundant components are identified and the independent components most representative of each paroxysmal event are selected and presented. The process of repetitively applying independent component analysis to a signal dataset is referred to herein as multitaper independent component analysis (MICA). The MICA technique will now be explained in more detail.

The MICA Technique

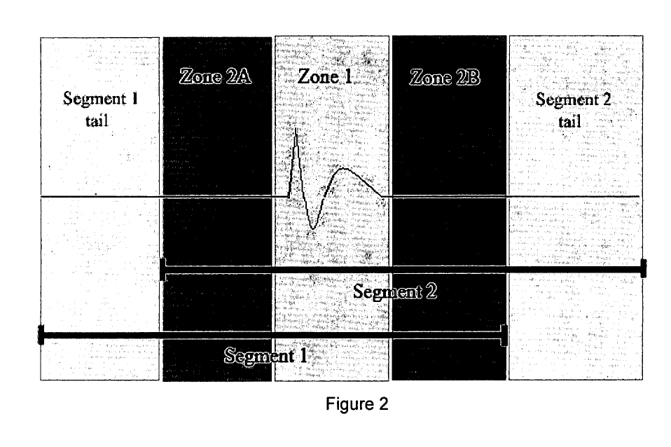

These limitations described above with regard to conventional applications of ICA can be partially overcome and ICA can be used for event extraction and presentation by using MICA. In this technique, ICA is performed repetitively on small portions of the EEG dataset and the independent components and corresponding scalp distributions produced are cross-multiplied to detect the appearance and disappearance of events in the record. This technique allows the length of each event to be approximated and an unlimited number of events to the detected. The scalp distribution of each event is also determined. The MICA method can be performed in several variations. Each of these variations will be explained. The first variation is illustrated in FIG. 2:

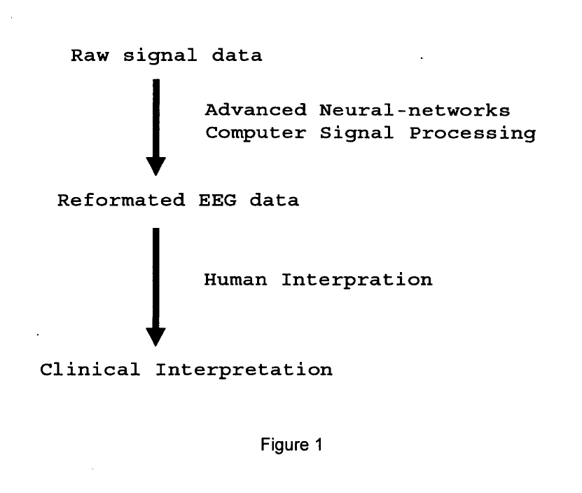

FIG. 2 illustrates an example of the isolation of a neurophysiologic signal event. In this example, an epileptiform spike is found in an EEG dataset containing 20 channels (produced from recording from 20 electrodes). Only one channel of the raw EEG dataset is shown in the diagram. ICA is performed on two segments of this EEG dataset, including all 20 channels and encompassing two different overlapping time periods of equal length, as illustrated by Segment 1 and Segment 2. In the example pictured in FIG. 2, both of these segments contain the spike event. ICA analysis of Segment 1 of the 20-channel dataset will produce 20 independent components and their respective scalp distributions. Likewise, ICA analysis of Segment 2 will also produce 20 independent components and their respective scalp distributions. In the set of 20 components and scalp distributions produced by ICA from Segment 1, one of the components will likely contain the spike event. In the set of 20 components and scalp distributions produced by ICA from Segment 2, one of the components will also likely contain this same spike event. Each of these 40 independent components (ICs) produced by performing ICA on these two segments can be divided into four zones: Zone 1, Zone 2A, Zone 2B, and tail zones (as in FIG. 1). Note that each of the three types of zones in both segments mark the same time span. The portions of each IC in Zone 1, Zone 2A, and Zone 2B are used in the calculation and this includes 120 IC fragments for each iteration (60 from Segment 1 and 60 from Segment 2). A numerical value, termed the paroxysmal event index (PEI) can be calculated using these IC fragments by this equation, using the summed products of the component fragments.
PEI=((2*(ΣICN1*ICM1))−((ΣICN2A*ICM2A)+(ΣICN2B*ICM2B))/LZ1
N=1 to the number of channels (for the first set of independent components)

M=1 to the number of channels (for the second set of independent components)

| | |
|---|---|
| ICN1 | IC number N from Segment 1, Zone 1 |
| ICN2A | IC number N from Segment 1, Zone 2A |
| ICN2B | IC number N from Segment 1, Zone 2B |
| ICM1 | IC number M from Segment 2, Zone 1 |
| ICN2A | IC number M from Segment 2, Zone 2A |
| ICN2B | IC number M from Segment 2, Zone 2B |
| LZ1 | length (in datapoints) of Zone 1 |

Σx*y denotes summation of the product of the amplitude of signal x and y at each time point

The PEI is calculated between each IC of the two segments, forming a matrix containing, in this example, 400 values. The PEI will be high at the matrix location in which the IC from Segment 1 containing the spike pattern and the IC from Segment 2 containing the same spike pattern are used in the equation, as long as the spike event falls in Zone 1. This is because when these two ICs are used to calculate the PEI, their Zone 1 segments will correlate strongly and their Zone 2A and Zone 2B segments will not.

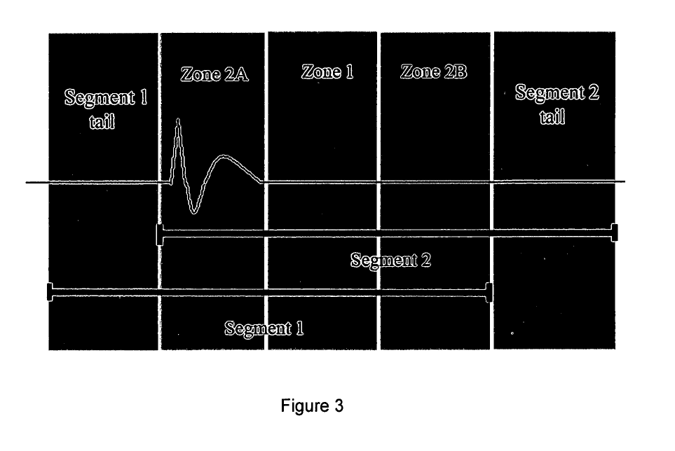

If, as in FIG. 3, a paroxysmal event falls within Zone 2A and not Zone 1 or Zone 2B, a small PEI will be produced because the summed product of the components containing paroxysmal event will be large in Zone 2A but not in Zone 1 or Zone 2B.

If, for example, a spike event longer than the length of Zone 1 falls within Zone 1, a smaller PEI will be produced by this equation, since the summed products of not only Zone 1 but also Zone 2A and Zone 2B will be increased, causing the PEI to be lower. If a spike event substantially shorter than Zone 1 falls within Zone 1, it is unlikely to produce a large PEI because it will not produce a high enough summed product in Zone 1 to outweigh the lack of a summed product in Zone 2A and 2B. In this way, the equation is sensitive to patterns resolved by ICA of approximately the length of Zone 1.

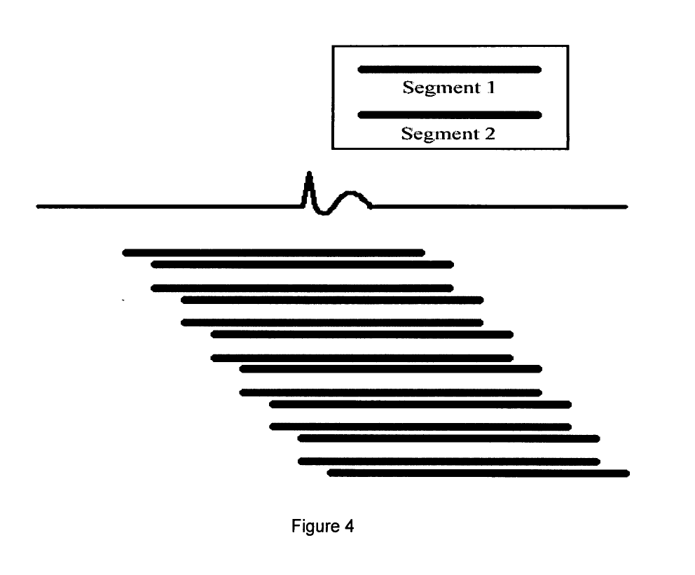

If this process is repeated throughout the dataset, using a Segment 1 and Segment 2 of the same length and overlapping in the same way, yet in different locations, the dataset can be scanned for patterns of the length of Zone 1. This is illustrated in FIG. 4. A high PEI will only be generated by the spike in FIG. 2 during the iteration in which the location of Segment 1 and Segment 2 are such that the spike falls in Zone 1 of both segments. If this spike falls in one of the other zones (as illustrated in FIG. 3), this spike event will not cause a significant elevation in the PEI value.

The length of Segment 1 and Segment 2 are made to be longer than the length of the three zones combined because it is believed that this improves the resolution of the paroxysmal event. This may be because it prevents the event from being overly fragmented into many ICs when ICA is performed on each segment. Through experience, it appears that best results are obtained using equal lengths of Zone 1, Zone 2A, and Zone 2B and using a tail length of three times the length of Zone 1. But these proportions are somewhat arbitrary and need to be optimized by further research.

Visualizing MICA Calculations

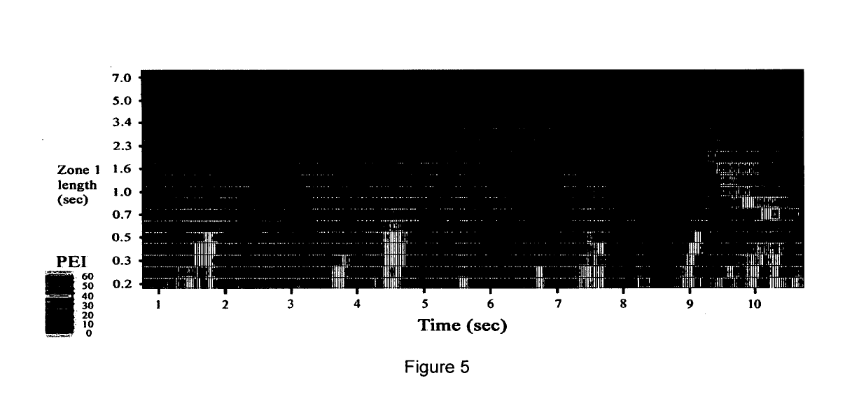

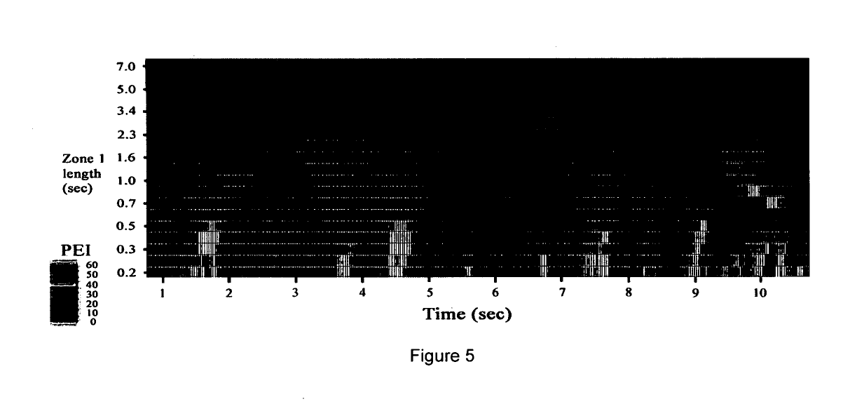

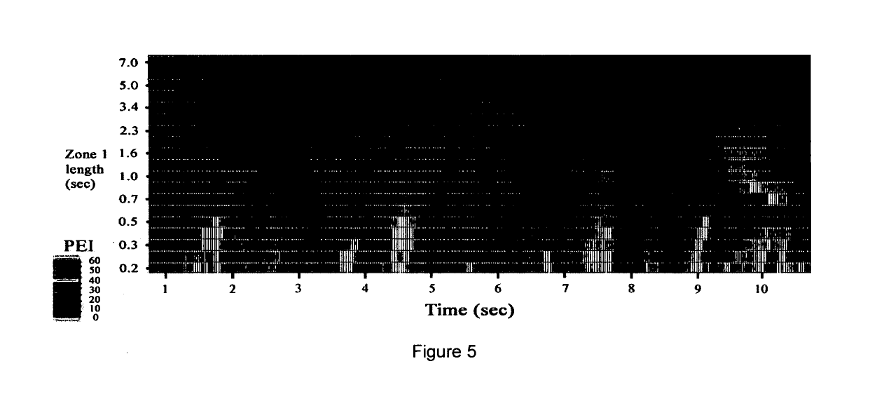

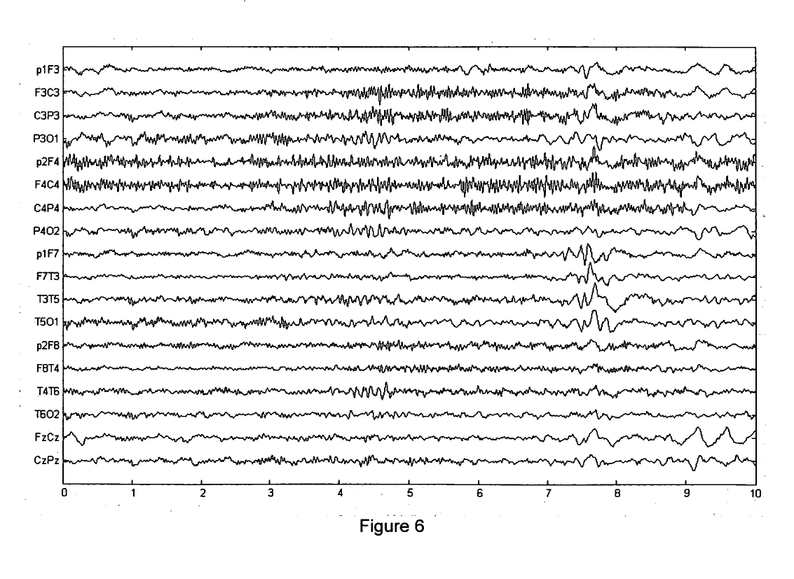

When a typical clinical EEG dataset is scanned for paroxysmal events using MICA, a plot displaying the maximum PEI value obtained for each Zone 1 length over time can be plotted in a graph. FIG. 5 displays 10 seconds of EEG data scanned using MICA, with time plotted on the x-axis, event length plotted on the y-axis (with larger lengths at the top of the graph) and maximum PEI displayed in color (red representing the highest PEI value). FIG. 6 is the seconds of raw EEG data displayed in an AP bipolar montage. In this example, a PEI value is calculated for every 1/6 Zone 1 length for 19 different Zone 1 lengths throughout the dataset. This increment of 1/6 Zone 1 length represents the temporal resolution of the MICA calculation and the choice of this value is also somewhat arbitrary. It is probably represents a higher resolution than is needed to identify most paroxysmal signal events, but has been used to make sure that no events are missed in this preliminary work.

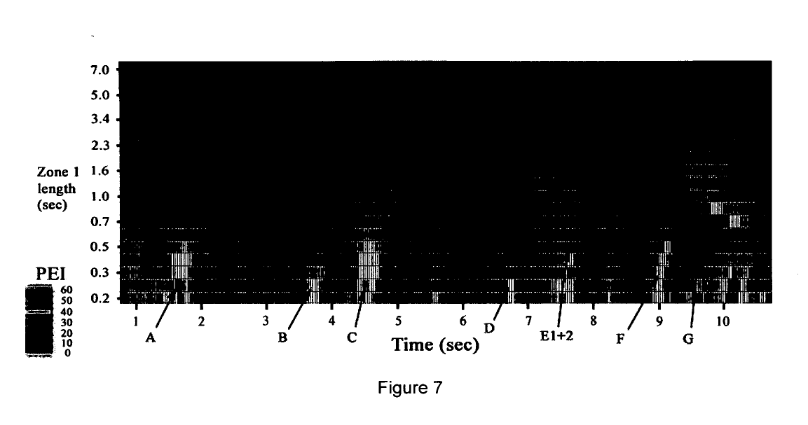

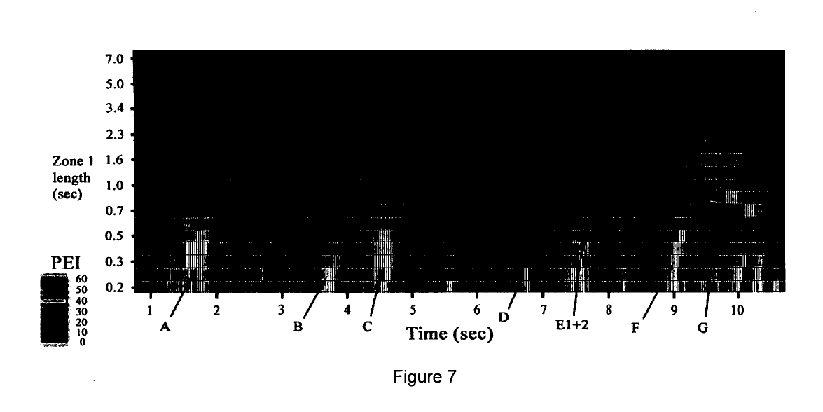

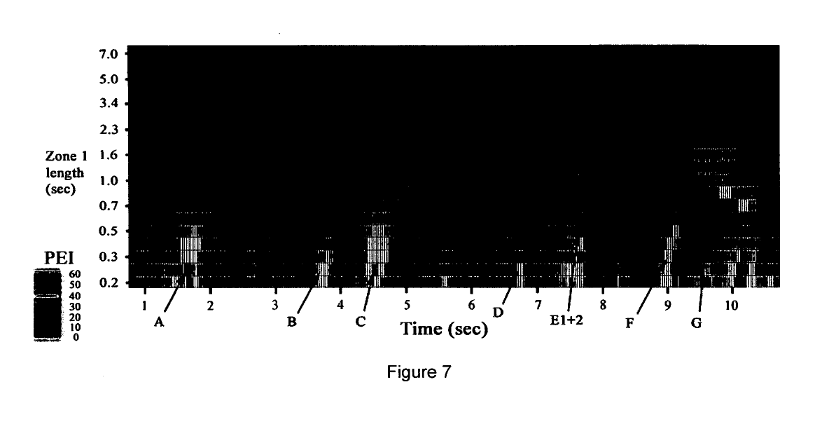

In FIG. 7, eight paroxysmal events detected by the MICA algorithm have been labeled by letters A-G.

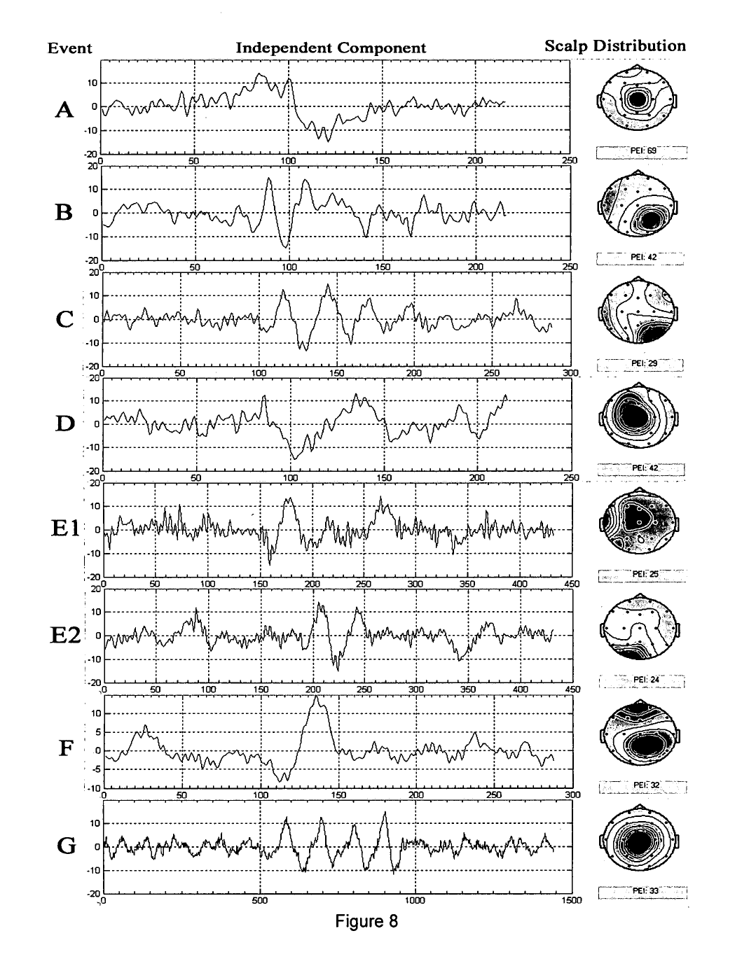

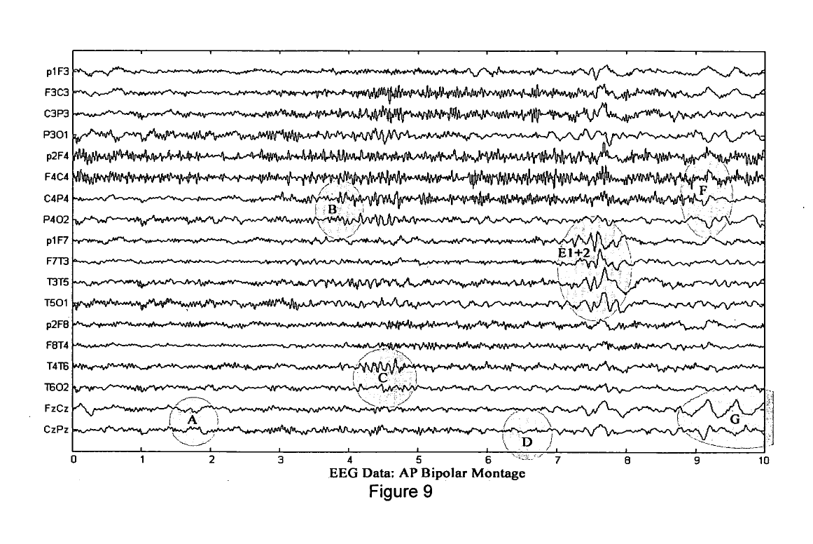

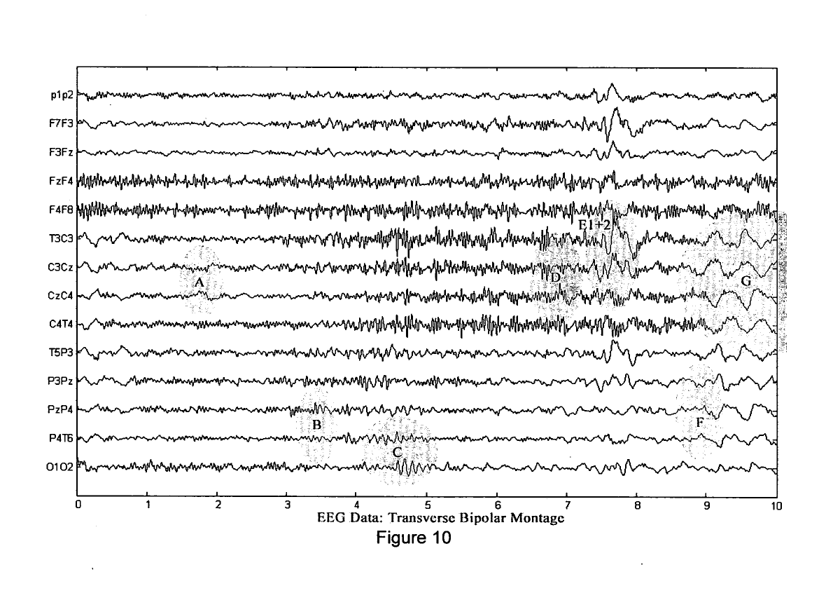

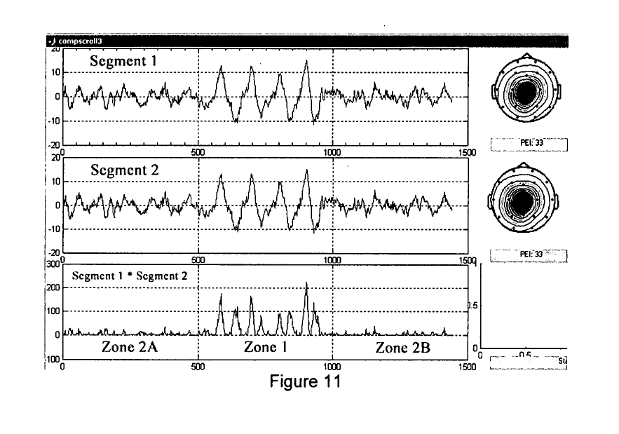

In FIG. 8, the eight independent components detected as events by the MICA method are displayed, with their respective scalp distributions. FIGS. 9 and 10 display some of the patterns within the raw EEG data (displayed in two different EEG montages and labeled with letters) which were detected by MICA as independent component events. FIG. 11 depicts the Zone 2A, Zone 1, and Zone 2B signals from two overlapping independent components which are cross-multiplied to form a third signal, pictured below the other two signals, as an illustration of the MICA method.

Could a Simpler PEI Calculation for MICA Work?

A simplified method of performing this analysis, which is only slightly less computationally intensive, is to examine each independent component and select the ones which have higher integrated amplitude in Zone 1 in comparison to Zone 2A added to Zone 2B. In this simpler method, the PEI would be calculated in this way:
PEI=ΣICN1−(ΣICN2A+ΣICN2B)/LZ1

N=1 to the number of channels (for the first and only set of independent components)

ICN1 IC number N from Segment 1, Zone 1

ICN2A IC number N from Segment 1, Zone 2A

ICN2B IC number N from Segment 1, Zone 2B

LZ1 length (in datapoints) of Zone 1

Σx*y denotes summation of the product of the amplitude of signal x and y at each time point

The disadvantage of calculating the PEI in this way is that it does not provide as much information as with the former method. For example, using the first (more complex) method for calculating PEI, for each IC in the first set of ICs, one IC may be identified which correlates well with one of the lCs from the second set of ICs. If both of these ICs, when multiplied together, produce a high PEI signal, these ICs will be selected, paired together, and stored as an event. But there is an important question: how much of that event is captured by these two ICs and their respective scalp distributions? Is it possible that there are other ICs in the two IC sets which contain information derived from this event? How can this be measured? Ideally, these two ICs should be very different from all of the other ICs in the two sets of ICs produced by the two ICA calculations. But are they? It may be possible to answer this question (to detect if one or both of the involved ICA calculations have produced ICs with this event well isolated in just one IC from each set or alternatively, scattered among many of ICs and not isolated into one IC from each ICA calculation): if the zone ICN1 from each component in ICA set 1 is cross-multiplied and summed with all of the zone ICM1 from each component in ICA set 2, this can produce a N by M matrix. Ideally, each column and row of this matrix should contain one high number and the rest low numbers. In general, it has been determined that the number representing the percentage of the sum of the three highest numbers of that column or row that the highest number composes (which is referred to herein as the fraction-of-top-three or FTTrow and FTTcolumn and especially the sum of the two FTTsum) is a useful measure for how well that event is ‘isolated’ by ICA. If an event is not ‘isolated’ well, it may be for several reasons:
(1) The ICA window for detecting the event may be too brief, causing the paroxysmal event of interest to be ‘fractured’ into many different ICs. If this event is more ‘fractured’ in one of the ICA windows more than the other, the FTTsum for that event will go down. This is a clue that it may be possible to ‘fracture’ this event into subcomponents. This suggests that smaller ICA windows as part of MICA analysis might be important to capture all of the ICs that could be isolated from this event.
(2) ICA may just not be able to resolve this event well due to a relentless ‘overcompleteness problem’ which is present even in a window of relatively short length (still too many source signals relative to detected signals even in a brief ICA window). This could also be due to an inadequate source-signal solution produced that particular ICA calculation. This would be important to know since it implies that the event detected has an undesirable quality and/or signal-to-noise ratio. But in any case, the first method for computing the PEI using two ICA windows has been the most interesting since it provides more information about the nature of the independent components produced.
   Additional Factors for Calculating the PEI

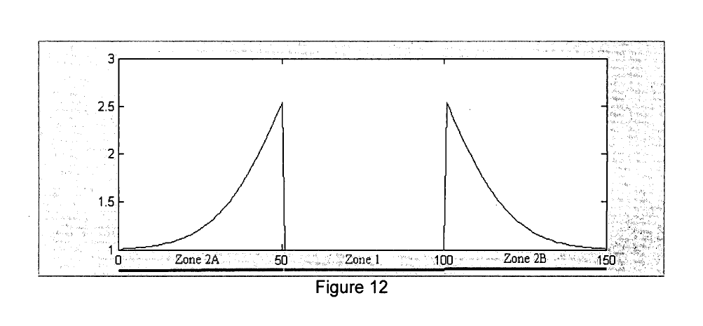

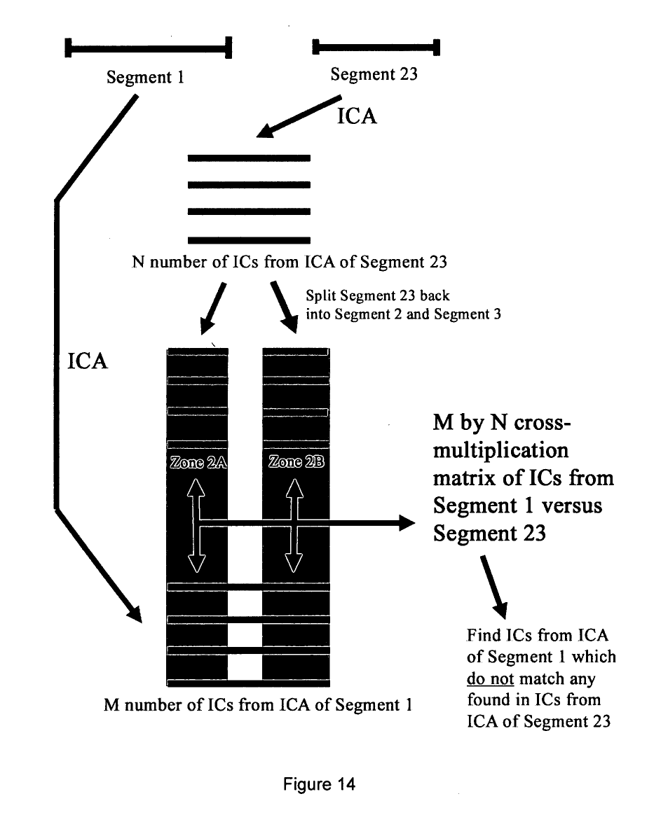

Calculating the PEI using the techniques above may not be sufficient. There are three additional problems:
(1) Events with higher amplitude will be detected to the exclusion of events of lower amplitude
(2) Events consisting of lower frequencies will be detected at the exclusion of events consisting of higher frequencies
(3) The length of each captured event cannot be easily specified due to so many independent components derived from slightly different temporal windows being detected and catalogued for each single event.
(4) In the PEI calculation using two independent components above, a mean component may be created from two independent components which, by chance, co-occur and resemble each other in their signal shape, but have very different scalp distributions.
   Each of these problems is addressed by modifying the independent components before they are analyzed, excluding some independent components from analysis, and/or by adding additional variables to the PEI equation:
(1) If only a high-amplitude electrographic event can produce a high PEI, and if the PEI is used as a method for deciding which independent components are stored as events and which are not, then low-amplitude events will be discarded by this technique. This would not be desirable since low-amplitude events may be just as paroxysmal as high-amplitude events and potentially just as clinically relevant. To compensate for this, all of the independent components are normalized before analysis such that their maximal signal amplitude is 15. (This is chosen arbitrarily.) The ability of this multitaper BSSS approach to detect paroxysmal events of relatively low signal strength is possibly its most interesting feature.
(2) Since the PEI calculation is made by cross-multiplying and summing two waveforms, this technique produces a higher summed-product for signals that resemble a half sine-wave then for signals resemble a sign wave of one or higher cycles. This means that paroxysmal signal events such as eye-blinks, which are monophasic, have a tendancy to be labeled with the highest PEI values while higher frequency paroxysmal signal events, such as vertex waves, are labeled with lower PEI values. To compensate for this, the PEI value is calculated with a ‘sign change factor’ (SCF) value as part of the equation. The SCF is computed using the number of times the independent component signal crosses the midline or zero-point (which is a crude measure of the peak signal frequency). This SCF factor is calculated in such as way that it is near one for peak frequencies in the one to two Hz range but approaches the number two with frequencies of four or higher. This is to help moderate-downward the high PEI values produced by such simple monphasic waveforms as eye blinks. FIG. 14 below is a plot of the SCF value for signal midline-crosses between the number of one and fifty.
   SCF=(0.5*(a tan(x−4)/(pi/2)))+1.5
   The final PEI value is modified by the SCF such that:
   PEI=PEI*SCF
(3) Since multiple independent components are detected for each event, using the MICA method a technique for determining which independent components are representing the same paroxysmal event and for selecting just one of them has been developed. This is term ‘redundancy reduction’. But to prevent too many independent components from be detected, the two independent components which are multiplied together and summed to calculate the PEI are also multiplied by a Gaussian-derived function in order to minimize the number of independent components there are produced by each event. This Gaussian function resembles a typical Gaussian curve in the regions that are multiplied with the Zone 2A and Zone 2B portions of the independent components. But in the center, in Zone 1, the function is set to be one. A plot of this function is presented in FIG. 12. The purpose of using this function in calculating the PEI is to decrease the PEI for those components that have the edges of the paroxysmal event falling into the regions of Zone 2A and Zone 2B immediately adjacent to Zone 1. This decreases the number of independent components which produce a high PEI value for a given paroxysmal event.
(4) In the PEI calculation using two independent components as described above, a mean component may be created from two independent components which, by chance, co-occur and resemble each other in their signal shape, but have very different scalp distributions. To prevent this, several steps are taken. First, the scalp distribution maps are normalized: each scalp distribution is set such that the maximal positive or negative component is set (somewhat arbitrarily) to 15 and the other components of the scalp distribution are proportioned accordingly. The number 15 was selected because this seemed to be the average maximal value for most scalp distributions produced by Infomax ICA. Secondly, independent components produced by PEI calculations that involve two independent components with scalp distributions that differ substantially are excluded and not placed into the independent component database. A ‘weights correlation matrix’ is formed by cross-multiplying and summing the scalp weights vectors of the two independent components that are being compared to produce the PEI. If this weights factor (WF) does not equal at least 200, then these independent components are discarded and not placed into the database.
   Component Technique for Detecting Events Using ICA

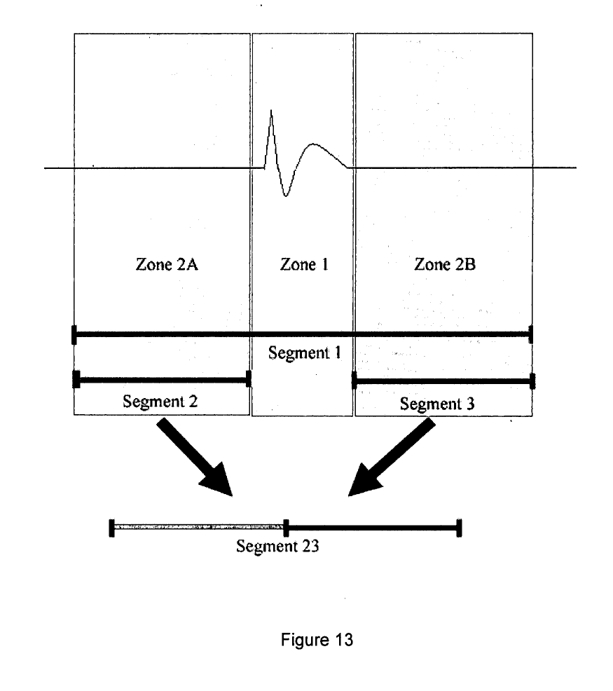

Another method for performing MICA is to use what is termed the ‘component technique’. This technique involves detecting events based on an ‘ICA window filter’. This technique is illustrated in the FIGS. 13 and 14 below. Again, multiple ICA analyses are performed at many lengths at each of many selected temporal locations within the dataset, in a multitaper method. As depicted in FIG. 13, at each time point selected during MICA analysis, two lengths of signal data are selected. One length, termed Segment 1, is selected to incorporate the whole amount of signal data within the window (all of Zones 1, 2A, and 2B). Another length, Segment 2, contains only the signal data in Zone 2A, and a third length, Segment 3, contains only the signal data in Zone 2B. A new segment, termed Segment 23, is created by concatenating Segment 2 and Segment 3. As depicted in FIG. 14, ICA is then performed on Segment 1 and Segment 23, producing two sets of ICs and their respective scalp distributions. The ICs produced by ICA of Segment 1 are each cross-multiplied with the ICs produced by ICA of Segment 23 (only over the temporally equivalent time points shared by Segment 1 and Segment 23), producing a summed cross-multiplication matrix similar to ones described previously. Any IC produced by ICA of Segment 1 which does not correlate with any ICs from Segment 23 is presumed to possibly contain an event. These ICs and their respective scalp distributions are stored, characterized, sorted, and presented to the neurophysiologist, as above.

MICA Event Storage and Characterization

In one implementation of the subject matter described herein, the independent components, their respective scalp distributions, and other information generated by the MICA software may be stored in databases. In one example, MICA can be implemented by storing this information in two different matrix databases. The first database is a matrix in which each element is itself a matrix. This may be implemented in the MATLAB software environment as a ‘matrix cell structure’ type matrix variable. Each row of this matrix contains a signal event which is the product of MICA analysis and contains columns for each of these types of data in matrix form:
(1) one-dimensional matrixes of independent component signals of different lengths (containing Zone 1, Zone 2A, and Zone 2B)
(2) one-dimensional matrices of numbers representing the temporal location of each datapoint in the independent component
(3) one-dimensional matrices of scalp distributions
(4) one-dimensional matrices of the FFT of the Zone 1 of each independent component
(5) one-dimensional matrices of the frequency points for each FFT calculation

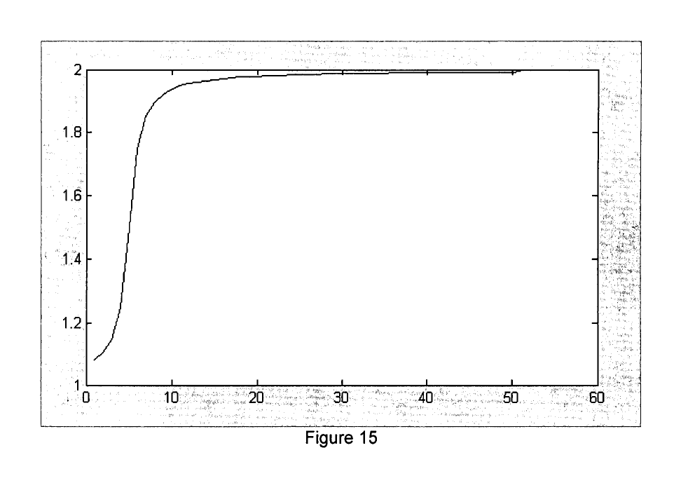

The second component of this database is a matrix of numbers. The rows represent each signal pattern that is captured. The columns represent characteristics of each signal event that is captured. In my implementation of MICA, there are 32 columns:
(1) The first column is a number which links each row to a specific row in the first cell of the database described above which contains the independent components, scalp distributions, and other matrices (in case this second numerical database is sorted). This is termed the “PatternLink” number.
(2) A column containing a number which represents a unique event which this independent component represents. This will be described further below.
(3) This column usually contains all ones. That is because only independent components that are unique are stored. The term ‘unique’ means that, when the two sets of overlapping components are compared, the independent component in set A is best correlated with an independent component in set B. But conversely, the independent component in set B is also best correlated with the independent component in set A. Independent components that are not ‘unique’ in this way are not stored for later analysis.
(4) This column is the PEI value for each component.
(5) The column stores the temporal location point at the center of this component.
(6) This column stores the length of the Zone 1 segment in datapoints.
(7) This column stores the temporal location of the beginning of Zone 2A
(8) This column stores the temporal location of the beginning of Zone 1
(9) This column stores the temporal location of the beginning of Zone 2B
(10) This column stores the temporal location of the end of Zone 2B
(11) This column stores the peak spectral frequency of Zone 1 (when a FFT is run on the signal in Zone 1 of the independent component).
(12) This column stores the maximum value found in the matrix of component weights (before normalization)
(13) This column stores the minimum value found in the matrix of component weights (before normalization)
(14) This column stores the ‘weights polarity,’ which is calculated as:
HWV=the highest value in the component weights matrix
LWV=the highest value in the component weights matrix
   WP=(HWV−LWV)/30
This is a measure of how big a difference there is between the most positive and most negative regions of the scalp map. The WP value approaches one at the maximum polarity difference in a scalp weights map.
(15) This column contains values which are termed the ‘sign change factor’ (SCF) values. As described above, this factor is used to make the PEI value calculation for each component. The SCF is computed using the number of times the signal crosses the midline (which is a crude measure of the peak signal frequency). This factor is near one for peak frequencies in the one to two Hz range but approaches the number two with frequencies of four or higher. This is to help moderate-downward the high PEI values produced by such simple monphasic waveforms as eye blinks. FIG. 15 is a plot of the SCF value for signal midline-crosses between the number of one and fifty.
   SCF=(0.5*(a tan(x−4)/(pi/2)))+1.5
(16) This column contains the fraction-of-top-three row (FTTrow) value as described above.
(17) This column contains the fraction-of-top-three column (FTTcolumn) value as described above.
(18) This column contains the fraction-of-top-three sum (FTTsum) value as described above.
(19) This column contains a value called the disproportionality index (DI). This is a measure of how disproportional the summed product of Zone 2A is in comparison to Zone 2B. An elevated Dl signals that the component captured by the MICA process may be only a fragment of the actual event. Sometimes when the end of a long event is found in Zone 2A and Zone 1 or the beginning of a long event is found in Zone 1 and Zone 2B, an event is captured but it is only a fraction of the true event. This is signaled by a high Dl. The Dl is calculated as described below:
ICN2A IC number N from Segment 1, Zone 2A
ICN2B IC number N from Segment 1, Zone 2B
ICN2A IC number M from Segment 2, Zone 2A
ICN2B IC number M from Segment 2, Zone 2B
Σx*y denotes summation of the product of the amplitude of signal x and y at each time point
   DI=abs(1−((ΣICN2A*ICM2A)/(ΣICN2B*ICM2B)))
   N=1 to the number of channels (for the first set of independent components)
   M=1 to the number of channels (for the second set of independent components)
(20) The last column contains the focal index (FI). This is calculated by dividing the value of the greatest (either positive or negative) value in the weights matrix by the next highest value in the weights matrix. This value indicates whether the weights distribution shows activation mainly at just one electrode, suggesting that the event detected is likely artifact.
   Redundancy Reduction

One of the technical challenges of using this technique is how to deal with the large number of independent components (and associated scalp distributions) produced by each signal event. That is, each event will usually produce multiple independent components (and associated scalp distributions), which are found to have a high PEI and are therefore captured and catalogued by MICA. This challenge has several facets:
(1) How do you store so many independent component events in a database?
(2) How do you determine which independent components are representations of the same event and are thus redundant?
(3) How can you use this ‘problem’ of redundancy to learn something about the events detected using this technique?

A solution for question one (1), as described above, is the utilization of a MATLAB cell matrix. The second (2) problem is more difficult. In order to decrease the redundancy in the dataset, the independent components are sorted based on their temporal center location in the dataset. Then, beginning sequentially with first occurring independent component, each independent component is compared with all other independent components which overlap that component temporally by at least 50% of the length of the shortest of the two overlapping independent components. The comparison is made by cross-multiplication and summation (similar to the way that the PEI is calculated). A pattern comparison index (PCI) is computed for the comparison of each overlapping independent component pair in the database. The PCI is calculated as below:

SP=summed product of overlapping regions of the two independent components

SL=the segment length (in datapoints) of the overlapping region of the two components
PCI=SP/SL
The first independent component in the sorted database is labeled as having a pattern number of one. All other independent components that overlap this component by at least 50% (of the smallest component) are compared with it. If the PCI for any of these comparisons is greater than 0.75 (a number that is chosen somewhat arbitrarily), then these independent components are also labeled with a pattern number of 1. Any components that overlap but do not have a PCI greater than 0.75 are given another pattern numbers (of two or greater). This is continued throughout the database of independent components until all independent components are labeled with pattern numbers. In my experience, each event is captured between one and approximately twenty times.

Once the independent components have been labeled with pattern numbers, a decision must be made as to which one of the group to keep and which ones to discard. This is not an easy decision, since often the different independent components for a pattern, while looking somewhat the same, are of different lengths and have slightly different scalp distributions. There are two possible solutions to this issue and the MICA method does not specify which is to be used:
(1) One method is to just pick one independent component out of each pattern-grouping based on various criteria which could include: how high the PEI is, how high the FTT sum is, how low the disproportionality index is, etc. Or one independent component could be selected out of the group by selecting the independent component that contained the greatest variance within its window of independent component analysis.
(2) Another, potentially more powerful method, is to create a hierarchal representation scheme for each pattern which would consist of several elements of the database: a longer independent component and several shorter independent components that this longer independent component can be broken down into—all with the same pattern number. This second scheme could be reveal more information about each pattern. For example, an epileptiform spike may be represented as a spike-wave component of a longer length plus its two associated subcomponents of the spike and the wave (each of which have a different time of occurrence and scalp distribution).
   Independent Component Inversion-Detection and Display

Portions of independent components that are associated with paroxysmal events may be displayed in any suitable manner that is beneficial to event identification. For example, software may be provided to display raw signal data with events highlighted as illustrated in FIGS. 9 and 10. Alternatively, the portions independent components that are associated with paroxysmal events may be displayed without the surrounding signal data, for example, as illustrated in FIG. 8.

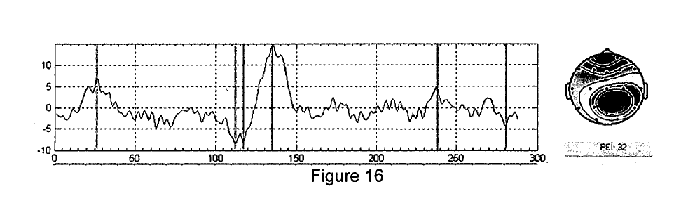

As part of the MICA technique, independent components and their scalp distributions need to be assessed individually to make sure that they have not been inverted during the ICA process. This can be accomplished by taking a number of timepoints within each independent component which have a relatively large value (either in the positive or negative direction, preferably a few of both), back projecting these time-points using the scalp distribution weights, and then comparing the produced raw data points to the actual raw data points in certain specific signal sources. So, first, in this method, as in FIG. 16, a number of timepoints within each independent component would be selected, based on their being the various “peaks” or “troughs”. In this figure these are marked with vertical green lines.

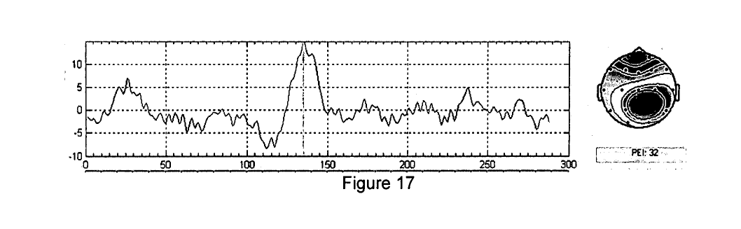

The value at these temporal points in the independent component (marked by the green vertical lines) are multiplied by the scalp weight of maximal positivity in the associated weights matrix. (In FIG. 16 this would be the weight representing Pz). If the independent component and scalp map have not been inverted by the ICA process, when these six amplitude values at these six time points produced by this back-projection are compared to the raw signal amplitude at the Pz electrode at these time points then the two calculations below should be true (or the IC has been inverted):
(1) The datapoints produced by the back projection which are peaks (lines 1, 4, and 5 in FIG. 16) should be close in amplitude to the signal amplitude of the raw data at the electrode with maximal weighting in the weight matrix at those time points represented by lines 1, 4, and 5.
(2) The datapoints produced by the back projection which are troughs (lines 2, 3, and 6 in FIG. 16) should be close in amplitude to the signal amplitude of the raw data at the electrode with maximal weighting in the weight matrix at those time points represented by lines 2, 3, and 6.
   This can be represented by an equation. If six time points are used for each component, an inversion detection factor (IDF) can be defined:
   ICp1=amplitude value of the independent component at peak time point 1
   ICp2=amplitude value of the independent component at peak time point 2
   ICp3=amplitude value of the independent component at peak time point 3
   ICn1=amplitude value of the independent component at trough time point 1
   ICn2=amplitude value of the independent component at trough time point 2
   ICn3=amplitude value of the independent component at trough time point 3
   Cmax=the raw signal channel that corresponds to the weight matrix position with the highest positive value
   Wmax=value of the weight matrix at Cmax
   Sp1=amplitude of raw signal at channel Cmax at the peak time point 1
   Sp2=amplitude of raw signal at channel Cmax at the peak time point 2
   Sp3=amplitude of raw signal at channel Cmax at the peak time point 3
   Sn1=amplitude of raw signal at channel Cmax at the trough time point 1
   Sn2=amplitude of raw signal at channel Cmax at the trough time point 2
   Sn3=amplitude of raw signal at channel Cmax at the trough time point 3 Dp1NI=abs((ICp1*Wmax)−Sp1) defines the difference between the projected Dp2NI=abs((ICp2*Wmax)−Sp2) peak value and the actual amplitude at the Dp3NI=abs((ICp3*Wmax)−Sp3) Cmax channel if the IC is not inverted Dp1I=abs((−1*ICp1*Wmax)−Sp1) defines the difference between the projected Dp2I=abs((−1*ICp2*Wmax)−Sp2) peak value and the actual amplitude at the Dp3I=abs((−1*ICp3*Wmax)−Sp3) Cmax channel if the IC is inverted Dn1NI=abs((ICn1*Wmax)−Sn1) defines the difference between the projected Dn2NI=abs((ICn2*Wmax)−Sn2) trough value and the actual amplitude at the Dn3NI=abs((ICn3*Wmax)−Sn3) Cmax channel if the IC is not inverted Dn1I=abs((−1*ICn1*Wmax)−Sn1) defines the difference between the projected Dn21=abs((−1*ICn2*Wmax)−Sn2) trough value and the actual amplitude at the Dn3I=abs((−1*ICn3*Wmax)−Sn3) Cmax channel if the IC is inverted
   IDF=(Dp1I+Dp2I+Dp3I+Dn1I+Dn2I+Dn3I)/(Dp1NI+Dp2NI+Dp3NI+Dn1NI+Dn2NI+Dn3NI)
   If the IDF is greater than one, then the independent component needs to be inverted. The weights matrix is not inverted. (One of the two has to be changed.) If the independent component does not need to be inverted, it is displayed as in FIG. 17.

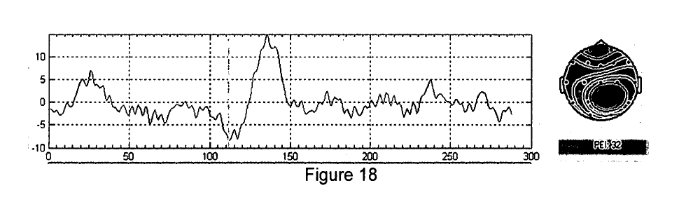

If the independent component has to be inverted, then it can be simply inverted and displayed with the yellow line in the same position as it is in FIG. 17, or it can be non-inverted and displayed as in FIG. 18. In this method of displaying independent components, the yellow line marks the point of maximal positivity in the raw data at that time point and at channel with the maximum positivity on the scalp weights map.

MICA Summary

In summary, the MICA technique can be viewed to include several stages:
(1) Multiple independent component analysis calculations in a multitaper method
(2) Detection of components with paroxysmal characteristics
(3) Signal inversion-detection and labeling
(4) Storage of components with paroxysmal characteristics as independent component events within a database of independent component events
(5) Redundancy reduction
(6) Further characterization of these stored independent component events

MICA is not a mathematical technique which seeks to improve the way that independent component analysis is performed, per se. To use an analogy, the MICA technique uses each independent component analysis calculation as a brick to create a wall. There are different types of ICA, including instantaneous methods (such as Infomax ICA) and summary methods. Infomax ICA was used in software that implements the subject matter described herein. Other methods for performing BSSS suitable for use with the subject matter described herein include the Hebbian learning algorithms, robust adaptive algorithms, and nonlinear principal component analysis. How BSSS is performed mathematically will likely continue change and improve. Any new and improved algorithms for making the BSSS calculation can easily be plugged into software that implements the subject matter described herein without modifying the multitaper BSSS algorithm described herein.

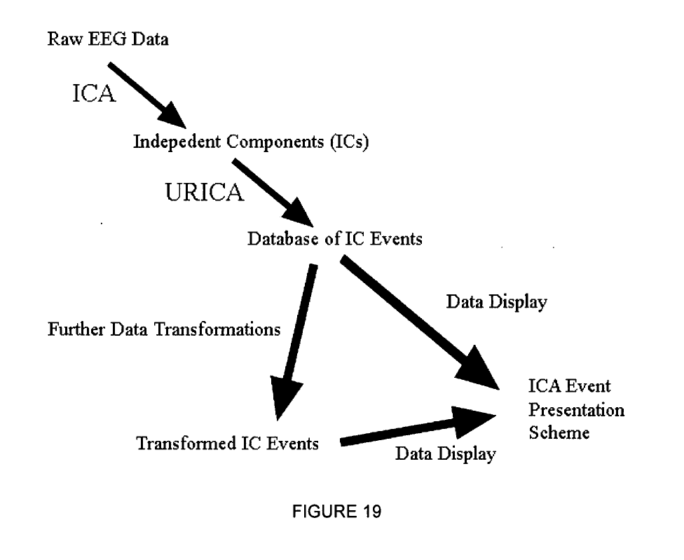

The database of independent components produced by MICA processing can be subjected to multiple further analyses. To name a few:
(1) Fourier transformation
(2) Wavelet transformation
(3) Three dimensional signal source (dipole) analysis
(4) “Spikiness” analysis
(5) Power analysis
   One of the main ideas behind the MICA technique is to develop a standard preprocessing procedure that transforms EEG or other data into independent component event space. After this MICA process is completed, characteristics of these independent component events can be plotted in a two dimensional plot with the x-axis being time, the y-axis being the length of the detected independent component, and the z-axis (color) representing the result of another type of analysis on these individual independent component events (such as peak spectral frequency, ‘spikiness’, power density, etc.) These other techniques for analysis can therefore be applied to the independent component events, instead of to the raw signal data. A general flow diagram for this is illustrated in FIG. 19. This would have several advantages:
(1) The signal data could be sorted and viewed in an intuitive scheme that could allow a clinician to look over a large amount of EEG data relatively quickly by looking at independent component event locations, lengths, and characteristics instead of at raw signal data.
(2) Artifactual signals would be separated from the electrocortical signals before other types of analysis (as listed above) are applied or before the database of events is viewed. Due to their unique characteristics, the many of the artifactual signals would likely cluster and be distinguishable from the electrocortical signals. (The artifactual signals may be able to be removed from the independent component event database by automated computer methods before visualization or further analysis.)
(3) Localization of events within the range of acquisition electrodes would be readily apparent to the clinician or researcher since scalp distribution maps would be displayed alongside all independent components. (This would make it easierfor medical personnel not as highly trained in electroencephalography to study signal data.)
(4) EEG events of relatively low amplitude, many of which are probably ignored by clinicians and researchers, could be detected consistently and thoroughly and databased for analysis and visualization.
   Other Potential Applications of Multitaper Blind Signal Source Separation

Although the multitaper BSSS technique has been developed to be used to enhance signal processing capabilities for neuroscience research and clinical applications, it could be applied to the analysis of any dataset of mixed signals which are originating from signal recording sources of known location. The multitaper BSSS method could be applied to the analysis of intracranial electroencephalographic data and magnetoencephalographic data. Other examples of biological data sets to which multitaper BSSS as described herein could be applied include a cardiac physiologic data set, for example, as output by an EKG machine. Examples of non-biological signal data sets to which multitaper BSSS could be applied include radar data sets, seismography data sets, and economic datasets.

While the computational demands of BSSS generally increase linearly based on the length of the analyzed signal, they increase exponentially based on the number of recorded signal channels. Since multitaper BSSS requires a large number of BSSS calculations, it is best suited for analyzing datasets which are very rectangular (long datasets with fewer signal sources) and least well suited for analyzing large datasets which are square (number of signal sources closer to the number of data points). So, it will be most useful for analyzing data recorded for a significant length of time from a limited number of recording devices but not useful for analyzing a shorter length of time of signal data from a large number of recording devices. A relatively square dataset does not have as much of an ‘overcompleteness’ problem as part of its BSSS solution. Rectangular datasets which could be analyzed with MICA include sonar datasets, radar datasets, seismography datasets, and economic datasets. The multitaper BSSS method could be implemented in software to allow computers to automate the capture and characterization of sonar events, seismography events, economic events, and other such events in any long rectangular signal dataset.

As described above, the subject matter described herein may be implemented using a computer program or programs that execute on one or more computing platforms. FIG. 19 is a block diagram of an exemplary system for identifying paroxysmal events using multitaper blind signal source separation analysis according to an embodiment of the subject matter described herein. Referring to FIG. 19, the system includes a multitaper blind signal source separation (BSSS) analysis engine 100 that receives signal data and repetitively applies blind signal source separation analysis to different time limited segments across the signal data in a multitaper method to extract components indicative of paroxysmal events. Multitaper BSSS analysis engine 100 may use any of the techniques described herein for identifying paroxysmal events from signal data. Multitaper BSSS analysis engine 100 may store the independent components in event database 102. A presentation engine 104 may extract paroxysmal event data from database 102 and may present the event data to a user. The event data may be presented in any suitable format that facilitates interpretation of the events, for example, as shown in FIGS. 8-10.

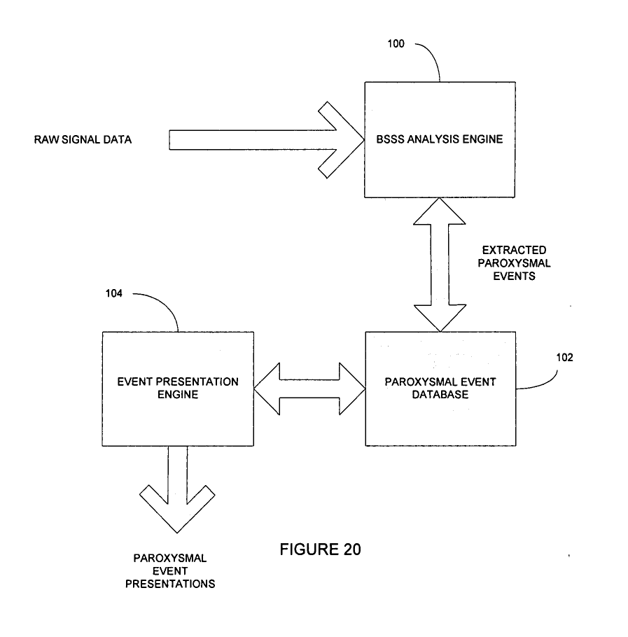

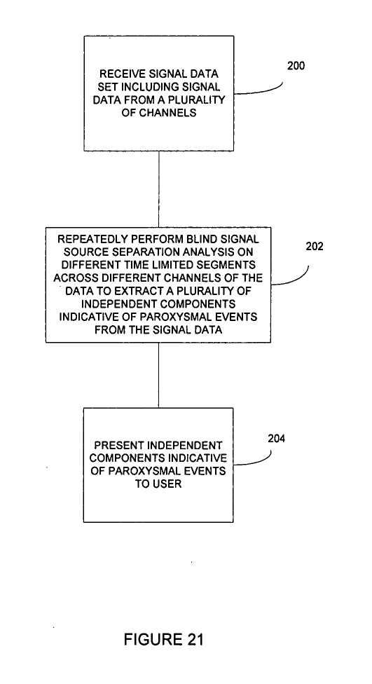

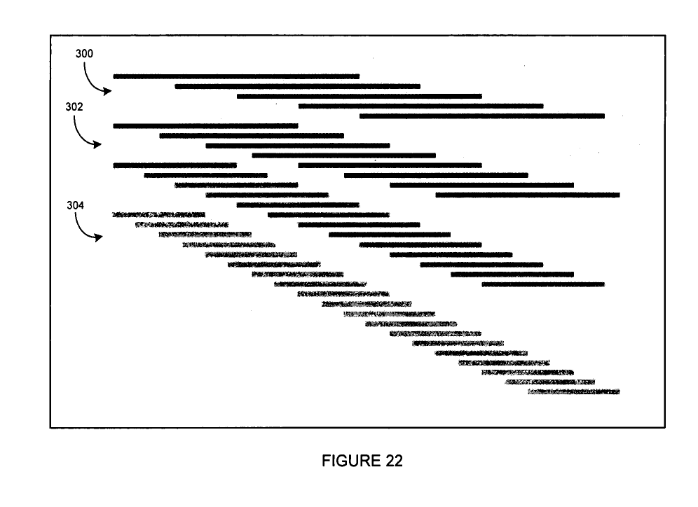

FIG. 21 is a flow chart illustrating exemplary overall steps that may be performed by the system illustrated in FIG. 20. Referring to FIG. 21, in step 200, a signal data set including signal data from a plurality of channels is received. For example, the signal data set may be received by BSSS engine 100 illustrated in FIG. 21. In step 202, blind signal source separation analysis is performed on different time limited segments across the signal data in a multitaper method to extract a plurality of independent components indicative of paroxysmal events from the signal data. For example, BSSS analysis engine 100 may repeatedly apply independent component analysis or another type of BSSS to different time limited segments in a multitaper method. An example of a multitaper method is illustrated in FIG. 22. FIG. 22 graphically illustrates a multitaper method suitable for use with the subject matter described herein. In FIG. 22, each set 300, 302, and 304 of horizontal lines represents overlapping segments of the same window size that are applied to a signal dataset, such as the dataset illustrated in FIG. 9. In the multitaper BSSS technique described herein, BSSS is performed on multiple overlapping segments of one window size and then performed on multiple overlapping segments of other window sizes, repetitively for many window sizes (usually 20-32) throughout the dataset. In FIG. 22, each set 300, 302, and 304 represents a given window length.

Returning to FIG. 21, in step 204, the components indicative of paroxysmal events are presented to a user. For example, presentation engine 104 illustrated in FIG. 19 may extract components indicative of paroxysmal events from database 102 and display the components to a user in a suitable format, such as those described above. Morphological characteristics, such as FFT spectra, spikiness, or other characteristics may also be presented to the user to increase the utility of the displayed data.

### REFERENCES

Bell A. J., Sejnowski T. J., “An information-maximization approach to blind separation and blind deconvolution,” 1995 Neural Computing, Vol 7, pp 1129-1159.
Makeig S., Jung, T. P., Bell, A. J., Sejnowski, T. J., “Blind separation of auditory event-related brain responses into independent components,” 1997 Proceedings of the National Academy of Sciences U.S.A., Vol 94, pp 10979-10984.
Makeig, S., Westerfield, M., Jung, T. P., Enghoff, S., Townsend, J., Courchesne, E., Sejnowski, T. J., “Dynamic brain sources of visual evoked responces,” Science 2002, Vol 295, Issue 5555, pp 690-694.
Kobayashi, K., Merlet, I., Gotman, J., “Separation of spikes from background by independent component analysis with dipole modeling and comparison to intracranial recording,” 2001 Clinical Neurophysiology, Vol 112, Issue 3, pp 405-413.
McKeown, M. J., Humphries, C., Iragui, V., Sejnowski, T. J., “Spatially fixed patterns account for the spike and wave features in absence seizures,” 1999 Brain Topography, Vol 12, Winter Issue 2, pp 107-116.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for paroxysmal event detection in signal data sets using multitaper blind signal source separation analysis, the method comprising:
(a) receiving a signal data set including signal data from a plurality of channels;
(b) repeatedly performing blind signal source separation analysis on different time limited segments across the signal data in a multitaper method to extract a plurality of components indicative of paroxysmal events from the signal data; and
(c) presenting the components indicative of paroxysmal events to a user.

2. The method of claim 1 wherein receiving a signal data set includes receiving a signal data set of biomedical signals.

3. The method of claim 2 wherein receiving a signal data set includes receiving an electroencephalographic (EEG) data set.

4. The method of claim 3 wherein receiving an EEG data set includes receiving an EEG data set where the signal data for each channel is collected using a scalp electrode.

5. The method of claim 3 wherein receiving an EEG data set includes receiving an EEG data set where the signal data for each channel is collected using an intracranial electrode.

6. The method of claim 2 wherein receiving a signal data set includes receiving one of a magnetoencephalographic data set and a cardiac neurophysiologic data set.

7. The method of claim 1 wherein receiving a signal data set includes receiving a non-biomedical data set.

8. The method of claim 7 wherein the non-biomedical data set includes a data set selected from a group consisting of a sonar data set, a seismography data set, a radar dataset, and an economic data set.

9. The method of claim 1 wherein repeatedly performing multitaper blind signal source separation analysis includes performing multitaper independent component analysis (MICA).

10. The method of claim 9 wherein repeatedly performing multitaper blind signal source separation analysis includes calculating a fast Fourier transform (FFT) FTT sum for pairs of components from different overlapping windows of blind signal source separation (BSSS) calculation, the FTT sum being indicative of the presence of common events occurring at the same time in the different windows of BSSS calculation and also being indicative of how well the pairs of components have been resolved.

11. The method of claim 1 wherein performing blind signal source separation analysis includes calculating paroxysmal event index (PEI) values for each of the components using the PEI values to extract the components indicative of paroxysmal events.

12. The method of claim 1 comprising processing the extracted components indicative of paroxysmal events to eliminate redundant events and storing the redundancy-processed components in a database.

13. A system for paroxysmal event detection in signal data sets using multitaper blind signal source separation analysis, the system comprising:
(a) a multitaper blind signal source separation analysis engine for receiving a signal data set including signal data from a plurality of channels and for repeatedly performing blind signal source separation analysis on different time limited segments throughout the signal data set using a multitaper method to extract a plurality of components indicative of paroxysmal events from the signal data; and
(b) a paroxysmal event presentation engine for presenting the components indicative of paroxysmal events to a user.

14. The system of claim 13 wherein the blind signal source separation analysis engine is adapted to process biological signal data.

15. The system of claim 14 wherein the biological signal data comprises electroencephalographic signal data.

16. The system of claim 13 wherein the multitaper blind signal source separation analysis engine is adapted to process non-biological signal data.

17. The system of claim 13 wherein the multitaper blind signal source separation analysis engine is adapted to apply independent component analysis to the time limited segments and thereby to extract the components indicative of the paroxysmal events.

18. The system of claim 17 wherein multitaper blind signal source separation analysis engine is adapted to compute a fast Fourier transform (FFT) sum for pairs of components from different overlapping windows of blind signal source separation (BSSS) calculation, the FFT sum being indicative of the presence of common events occurring at the same time in the different windows of BSSS calculation and also being indicative of how well the pairs of components have been resolved.

19. A computer program product comprising computer-executable instructions embodied in a computer-readable medium for performing steps comprising:
(a) receiving a signal data set including signal data from a plurality of channels;
(b) repeatedly performing blind signal source separation analysis on different time limited segments across the signal data in a multitaper method to extract a plurality of components indicative of paroxysmal events from the signal data; and
(c) presenting the components indicative of paroxysmal events to a user.

20. The computer program product of claim 19 wherein receiving a signal data set includes receiving an electroencephalographic data set.
